(19) United States
(12) Patent Application Publication
(10) Pub. No.: US 2012/0103822 A1
(43) Pub. Date: May 3, 2012

(54) ELECTROADDRESSING AND IN-FILM BIOPROCESSING USING STIMULI-RESPONSIVE HYDROGEL-FORMING POLYMERS

(75) Inventors: Xiao-Wen Shi, College Park, MD (US); Yi Liu, Bethesda, MD (US); Gregory F. Payne, Hunt Valley, MD (US); Xiaohua Yang, Ardmore, OK (US)

(73) Assignee: UNIVERSITY OF MARYLAND COLLEGE PARK, College Park, MD (US)

(21) Appl. No.: 13/266,433

(22) PCT Filed: Apr. 27, 2010
(86) PCT No.: PCT/US10/32575

§ 371 Date: Jan. 11, 2012

Related U.S. Application Data
(60) Provisional application No. 61/173,197, filed on Apr. 27, 2009.
(60) Provisional application No. 61/258,401, filed on Nov. 5, 2009.

(51) Int. Cl. C25D 9/02; G01N 33/53; C12N 13/00

(52) U.S. Cl. 205/317; 435/173.1; 435/7.1

(57) ABSTRACT

Methods for the generation of hydrogels formed by electrodeposition of an electroaddressable polymer are described. The hydrogels may contain one or more cell populations electroaddressed or electroaddressable to a location within the hydrogel and where the cells of the cell populations are entrapped by the hydrogel and are capable of expansion within the hydrogel and may be releasable from the hydrogel. Further provided are electroaddressable polysaccharide blends for the in-film expansion of a cell population, allowing probing of the cells and formation of immunocomplexes. Further provided are methods of using hydrogels containing electroaddressed or electroaddressable cell populations in in-film bioprocessing methods such as cell-based biosensing, protein-based biosensing, and in studies of cell signaling.

### CROSS-REFERENCE TO RELATED APPLICATIONS

This application claims the benefit of priority of U.S. Provisional Patent Application Ser. No. 61/173,197 filed Apr. 27, 2009 in the names of Xiao-Wen Shi, Yi Liu, and Gregory F. Payne for “ELECTROADDRESSING OF CELL POPULATIONS BY CO-DEPOSITION WITH CALCIUM ALGINATE HYDROGELS” and further claims the benefit of U.S. Provisional Patent Application No. 61/258,401 filed Nov. 5, 2009, in the names of Xiao-Wen Shi, Yi Liu, Gregory F. Payne, and Xiaohua Yang for “ELECTROADDRESSING OF CELL POPULATIONS BY CO-DEPOSITION WITH CALCIUM ALGINATE HYDROGELS AND IN FILM BIOPROCESSING OF THE SAME.” The disclosures of said U.S. Provisional Patent Application Nos. 61/173,197 and 61/258,401 are hereby incorporated herein by reference in their respective entireties, for all purposes.

### GOVERNMENT RIGHTS IN THE INVENTION

This invention was made with government support under Contract No. EFRI-0735987 awarded by the National Science Foundation and Contract No. W91B9480520121 awarded by the Department of Defense, Defense Threat Reduction Agency. The invention was also made with support under Contract No. CBET-0650650 awarded by the R.W. Deutsch Foundation. The government has certain rights in the invention.

### FIELD OF THE INVENTION

The present invention relates to the generation of hydrogels by electrodeposition of an electroaddressable polymer and to use of the same. The invention relates to the electroaddressing of cell populations within a biopolymer hydrogel where a cell population is entrapped by the hydrogel. The invention also relates to inclusion of additional stimuli-responsive polymers in the hydrogel. The invention additionally relates to methods of using such hydrogels in methods of cell-based biosensing, in the study of cell-cell signaling and in in-film bioprocessing.

### DESCRIPTION OF THE RELATED ART

Recent advances in genomics and proteomics relied upon the development of methods for the spatially-selective coupling of nucleic acids and proteins to specific “address” locations. There is a similar interest in developing methods to assemble prokaryotic and eukaryotic cells at specific addresses for applications that range from fundamental study of cell-cell signaling to high throughput screening and biosensing. Current methods to assemble cells at specific addresses include: selective adhesion of cells onto patterned 2D surfaces, photolithographic polymerization to entrap cells within 3D hydrogel networks, and printing methods that deliver cell suspensions or cell suspensions plus components that promote gel formation. In addition, microfluidic devices have been designed that enable addressing or immobilization of cells within specific compartments. Thus, a variety of methods have been developed to address and cultivate cells in array and microfluidic formats. Nevertheless, the search continues for simpler, generic, less expensive and more benign methods for cell addressing.

Traditionally, microbiologists have employed stimuli-responsive hydrogel-forming polysaccharides for cultivation and these polymers are also being extended to array and microfluidic formats. For instance, agar is a thermally-responsive polysaccharide that is routinely used in microbiology and recently, cell arrays have been printed onto or within agar gels. Alginate is an acidic polysaccharide that forms a gel in the presence of calcium ions. Calcium alginate gels are widely used to entrap and immobilize prokaryotes and eukaryotic cells, and recent research has extended the use of alginate to entrap cells and nanoparticles at the microscale. In addition, cell (or nanoparticle) containing alginate beads, bars, tubes and multi-lamellar films have been generated in microfluidic systems by controlling the contacting of streams containing alginate and calcium ions. Hydrogel-forming polysaccharides may offer advantages for cultivation in array and microfluidic formats since they are familiar surfaces/matrices, they form gels under relatively mild conditions, and gel-formation is reversible allowing the entrapped hydrogel contents (e.g., cells) to be liberated intact. Therefore hydrogel films are potentially useful for replicating biological microenvironments and preserving labile biological functions (e.g., to maintain cell viability).

Fabrication methods for film patterning often enlist convenient, spatiotemporally-controllable stimuli. For instance, printing and photolithographic patterning employ mechanical and optical inputs, while there are growing efforts to use electrical stimuli to perform functions such as electroaddressing. Electroaddressing is particularly attractive for assembly because it employs the capabilities of microfabrication to create surfaces that can impose spatiotemporally-controlled electrical signals.

Recently, some stimuli-responsive polysaccharides have been observed to be capable of electrodepositing at electrode surfaces in response to localized electrical signals (Wu, L. Q. et al., Langmuir 2002, 18, 8620; Yi, H. M. et al. Biomacromol. 2005, 6, 2881). In most cases, these polysaccharides electrodeposit in response to electrochemically induced pH gradients that neutralize the polymer. Chitosan was the first hydrogel-forming polysaccharide to be electrodeposited (L. Q. Wu, et al., Langmuir 2002, 18, 8620; X. Pang, I. Zhitomirsky, Mat. Chem. and Phys., 2005, 94, 245; X. L. Luo, et al., Anal. Biochem. 2004, 334, 284). Chitosan was shown to gel at the cathode surface in response to a localized high pH (R. Fernandes, et al., Langmuir, 2003, 19, 4058; Pang, X. and Zhitomirsky, I., Mat. Chem. and Phys. 2005, 94, 245). Mechanistically, the pH-responsive chitosan is induced to undergo a sol-gel transition in response to the localized region of high pH established at the cathode surface. Once electrodeposited at the cathode, the chitosan hydrogel film is stable in the absence of an applied voltage, provided the pH is retained above its pKa (˜6.5; chitosan re-dissolves at pH below its pKa).

Recently, Cheong and Zhitomirsky (M. Cheong, I. Zhitomirsky, Colloid Surf. A-Physicochem. Eng. Asp. 2008, 328, 73) reported the anodic electrodeposition of alginic acid for the generation of composite films. They proposed a mechanism in which the localized low pH at the anode resulted in a neutralization of sodium alginate to alginic acid and neutralization of this polysaccharide resulted in gel formation. While this electrodeposition method is appropriate for the generation of composite materials, the low pH required to maintain the alginic acid gels may limit its use for culturing cells.

Therefore biological materials and mechanisms may offer opportunities to “biofabricate” functional hydrogel films.

Consequently improvements in the development and use of hydrogels for the electroaddressing of cell populations and in-film bioprocessing of the resulting hydrogels remain needed in the art. The present invention provides such hydrogels and methods of use thereof.

### SUMMARY OF THE INVENTION

The present invention relates generally to hydrogels formed by electrodeposition of one or more electroaddressable polymers. Such hydrogels may have one or more cell populations specifically electroaddressed therein. The invention also generally relates to hydrogels with one or more stimuli-responsive polymers integrated therein. The invention also provides method of use of hydrogels of the invention.

In one aspect, the invention relates to a method of forming a calcium alginate hydrogel on a substrate, comprising co-depositing a source of calcium ions and a source of alginate in the presence of an electrochemically charged anode under conditions such that a calcium alginate gel is electrochemically deposited on the substrate in the location of the anode.

In another aspect the invention relates to a method of forming a calcium alginate hydrogel on a substrate, comprising co-depositing a source of calcium ions, a source of alginate and a source of a stimuli-responsive polymer in the presence of an electrochemically charged anode under conditions such that a calcium alginate gel is electrochemically deposited on the substrate in the location of the anode and the polymer is contained within the calcium alginate gel. In one aspect the polymer is agarose.

In another aspect, the invention relates to a method of forming a calcium alginate hydrogel with one or more cell populations on a substrate, comprising co-depositing a source of calcium ions, a source of alginate, a source of a stimuli-responsive polymer and a first cell population in the presence of a first electrochemically charged anode under conditions such that a calcium alginate gel containing the first cell population is electrochemically deposited on a first location on the substrate. In one aspect the polymer is agarose.

In another aspect, the invention relates to a method of forming a calcium alginate hydrogel with one or more cell populations on a substrate, comprising co-depositing a source of calcium ions, a source of alginate and a first cell population in the presence of a first electrochemically charged anode under conditions such that a calcium alginate gel containing the first cell population is electrochemically deposited on a first location on the substrate. In a further aspect the method comprises additionally co-depositing a source of calcium ions, a source of alginate and a second cell population in the presence of a second electrochemically charged anode under conditions such that a calcium alginate gel containing the second cell population is electrochemically deposited on the substrate in a second location on the substrate.

A still further aspect of the invention relates to systems comprising a hydrogel generated by any of the methods of the invention. Such systems include, but are not limited to microfluidic systems and arrays.

Another aspect of the invention relates to a method of cell-based biosensing, comprising addition of a signaling molecule to a calcium alginate hydrogel comprising a first cell population entrapped therein, wherein the entrapped cells detect the signaling molecule and respond to the presence of the signaling molecule.

Another aspect of the invention relates to a method of forming a hydrogel on a substrate, comprising co-depositing at least two stimuli-responsive polymers, wherein at least one of the stimuli-responsive polymers is an electroaddressable polymer and wherein a second stimuli-responsive polymer is responsive to at least one of: thermal changes, pH changes, and electrical signals, under conditions such that a hydrogel comprising the at least two stimuli-responsive polymers is electrochemically deposited in the substrate.

Still another aspect of the invention relates to a hydrogel comprising at least two stimuli-responsive polymers, wherein at least one of the stimuli-responsive polymers is an electroaddressable polymer and wherein a second stimuli-responsive polymer is responsive to at least one of: thermal changes, pH changes, and electrical signals.

A further aspect of the invention relates to a method of in-film bioprocessing, comprising: electrodepositing a hydrogel as described herein, comprising at least a first cell population, wherein the first cell population comprises cells capable of expressing a surface expressed protein; proliferating the cells in the hydrogel; inducing expression of the surface expressed protein; adding an immunoreagent; and determining occurrence of binding of the immunoreagent and the surface expressed protein to form an immunocomplex. In one aspect the addition of an immunoreagent comprises electrophoresis.

Other aspects, features and embodiments of the invention will be more fully apparent from the ensuing disclosure and appended claims.

### BRIEF DESCRIPTION OF THE DRAWINGS

FIG. 1 provides the results of electrodeposition of calcium alginate gel on an ITO-coated glass slide (a) Front and (b) side views of a gel electrodeposited for 5 min at a current density of 3 A/m² (c) Calcium alginate hydrogel peeled from the slide. (d) Hydrogel after treatment with 0.1 M HCl to solubilize entrapped CaCO₃. (e) Thickness of dried calcium alginate films that had been electrodeposited for varying times.

FIG. 2 demonstrates the spatial selectivity of calcium alginate electrodeposition on a patterned chip. (a) The chip is a silicon wafer with two patterned gold electrodes (0.25 mm and 1 mm width). (b) Fluorescence photomicrograph of electrodeposited calcium alginate gels with entrapped fluorescent microparticles. (c) Confocal fluorescence image from the middle depth of the alginate gel with entrapped fluorescent microparticles.

FIG. 3 illustrates the growth of entrapped E. coli cells co-deposited with the calcium alginate hydrogel. (a) Growth curve as measured by optical density for cells incubated at 37° C. (b) The photographs of the inoculated and control alginate films after incubation for 24 h. (c) Bright field image of cell colonies (20-30 μm) after incubation for 7 hours in the alginate gel.

FIG. 4 shows the induction of entrapped E. coli cells to express the green fluorescence protein (GFP). (a) Time-course for the appearance of fluorescence after IPTG induction. (b) Fluorescence photomicrograph of induced E. coli cells entrapped within the alginate gel.

FIG. 5 illustrates the electroaddressing of two different E. coli populations to patterned ITO electrodes. (a) Schematic illustrating the sequential co-deposition of cells that express RFP (left electrode) and GFP (middle electrode) followed by the deposition of a control alginate gel (right electrode). (b) Photograph of patterned slide after sequential deposition. (c-d) Fluorescence photomicrographs of the patterned slide after IPTG induction using red or green filters. (e) Composite image of red and green fluorescence.

FIG. 6 illustrates the liberation of calcium alginate entrapped cells using sodium citrate. (a) Alginate entrapped, GFP-expressing E. coli cells before adding citrate. (b) Partial dissolution of calcium alginate gel after 2 min incubation with citrate. (c) Nearly complete dissolution of alginate gel after 5 min incubation with citrate. (d) Fluorescence image of liberated cells.

FIG. 7 shows alginate entrapped reporter cells that respond to autoinducer 2 (AI-2) by expressing GFP. (a) Fluorescence image of alginate-entrapped reporter cells (MDAI2(pCT6+pET-GFPuv)) incubated with added AI-2. (b) Control of alginate-entrapped reporter cells incubated in the absence of AI-2. (c) Control of alginate-entrapped non-reporter cells (MDAI2) incubated in the absence of AI-2.

FIG. 8 demonstrates the signaling between alginate-entrapped reporter cells and an external cell population. (a) Fluorescence image of alginate-entrapped reporter cells (MDAI2(pCT6+pET-GFPuv)) co-cultured with BL21 E. coli that can synthesize and secrete AI-2. (b) Control of alginate-entrapped reporter cells co-cultured with BL21 luxS⁻ E. coli that lack the ability to synthesize and secrete AI-2 (c) Control of alginate-entrapped non-reporter cells (MDAI2) co-cultured with BL21 E. coli.

FIG. 9 provides Scheme 1, which illustrates the mechanism for calcium alginate electrodeposition. The pH gradient at the anode triggers calcium release from insoluble CaCO₃ and this induces the localized gelation of calcium alginate at the anode surface.

FIG. 10 shows the results of electrophoresis performed on gels prepared according to Example 10, with differing levels of alginate (alg) and low melting agarose (aga) with protein markers (M) and fluorescently-labeled antibody (A).

FIG. 11 provides EQCM results from (a) in situ and (b) ex situ experiments to evaluate electrodeposition, as described in Example 8; (c) a photograph of a chip with patterned electrode addresses used to examine spatiotemporal control of deposition, as described in Example 9; and (d) results of sequential deposition as described in Example 9 with averaged fluorescence intensities listed for each deposit.

FIG. 12A provides a Yeast (VLRB.CT.Mut5) model; FIG. 12B provide photographs and photomicrographs of film grown yeast, as described in Example 10; FIG. 12C provides a growth curve for film-cultivated yeast, as described in Example 10.

FIG. 13 provides a schematic illustration of the sequence used in Example 11 to grow, induce, release and analyze VLR expression of film-cultured yeast; FIGS. 13B and 13C are confocal laser scanning microscopy images of yeast induced to express and display VLR protein; FIG. 13D provides graphs of the binding of the fluorescently labeled HEL antigen and the anti-HA immunoreagent over time, as described in Example 11.

FIG. 14 provides a schematic illustration of the sequence used in Example 12 to grow, induce, and analyze entrapped yeast in the absence of electrophoresis; FIG. 14B is confocal laser scanning microscopy images of film-entrapped yeast immunoanalyzed without electrophoresis; FIG. 14C provides a schematic illustration of the sequence used in Example 12 to contact entrapped yeast with anti-HA antibody using electrophoresis; FIG. 14D is confocal laser scanning microscopy images of film-entrapped yeast immunoanalyzed with electrophoresis.

### DETAILED DESCRIPTION OF THE INVENTION AND PREFERRED EMBODIMENTS THEREOF

The present invention relates to formation of hydrogels by electrodeposition of electroaddressable polymers, wherein such hydrogels optionally contain one or more cell populations electroaddressed or electroaddressable within the hydrogel and to use of the resulting hydrogels. Electroaddressing of biological components at specific device addresses is attractive because it enlists the capabilities of electronics to provide spatiotemporally controlled electrical signals. Furthermore, the hydrogels may optionally contain one or more additional stimuli-responsive polymers useful in in-film bioprocessing.

In one embodiment of the invention calcium alginate hydrogels are generated at specific electrode addresses by electrodeposition. The method employs the low pH generated at the anode to locally solubilize calcium ions from insoluble calcium carbonate. The solubilized Ca²⁺ can then bind alginate to induce this polysaccharide to undergo a localized transition from a suspension of solid particles in a liquid (sol) to an apparent solid, jelly-like material (gel) (a “sol-gel transition”). Calcium alginate gel formation is shown to be spatially controlled in the normal and lateral dimensions.

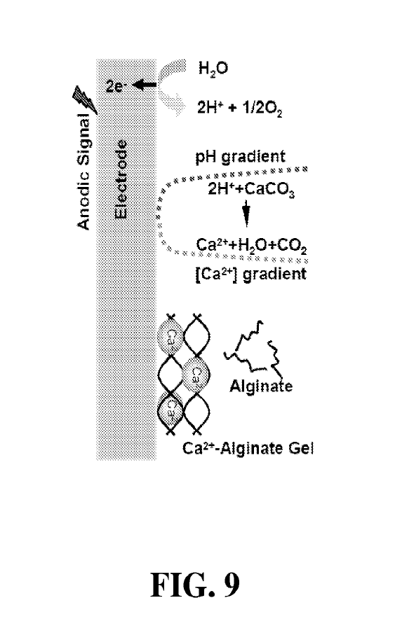

The scheme shown in FIG. 9 illustrates the mechanism for calcium alginate electrodeposition. The deposition solution contains soluble sodium alginate plus insoluble calcium carbonate (CaCO₃). Electrochemical decomposition of water at the anode leads to a pH gradient with a locally high proton concentration near the anode surface. This localized low pH triggers calcium release by the “solubilization” reaction:

CaCO₃+2H⁺→Ca²⁺+H₂O+CO₂

The acid-triggered Ca²⁺-solubilization reaction is further driven toward completion by the removal of CO₂ into the gas. The locally released Ca²⁺ is then free to interact with the alginate chains to generate the electrostatically-crosslinked hydrogel network.

Example 1 demonstrates calcium alginate electrodeposition and Example 2 demonstrates the spatial selectivity of calcium alginate electrodeposition.

In one embodiment the invention provides a calcium alginate hydrogel and a method for making the same.

Generally the application provides hydrogels formed by electrodeposition of an electroaddressable polymer. While hydrogels comprising alginate and calcium alginate are exemplified herein, it is understood that such electroaddressable polymers are exemplary polymers and that other such electroaddressable polymers, e.g. chitosan, are contemplated in hydrogels of the invention. As described herein, an electroaddressable polymer is a polymer which, through the operation of electrical signals, can be placed in a desired position on a substrate. Accordingly electroaddressable polymers are stimuli-responsive polymers, where the stimulus is an electrical signal or the stimulus is controlled by an electrical signal (e.g., pH gradient or solubilization of calcium ions).

The deposition method used to form hydrogels of the invention is sufficiently benign that it can be used to entrap cells. The entrapped cells are able to grow and respond to chemical inducers in their environment. Also, the entrapped cells can be liberated from the gel network. Where the hydrogel is a calcium alginate hydrogel, the entrapped cells can be liberated by adding sodium citrate that can compete with alginate for Ca²⁺ binding.

Cells or populations of cells that may be trapped in hydrogels of the invention are cells that may be detected, examined, induced, proliferated, tested, quantified, or otherwise evaluated within the hydrogel. The cells or populations of cells may also encompass reporter cells or signaling cells. Within the hydrogel, the population of cells remains viable, and can proliferate, respond to the environment, and can even be released from the hydrogel under appropriate conditions, as releasably entrapped cellular species.

The capabilities of calcium alginate electrodeposition are illustrated by entrapping reporter cells that can recognize the quorum sensing autoinducer 2 (AI-2) signaling molecule. These reporter cells were observed to recognize and respond to AI-2 generated from an external bacterial population. Thus, calcium alginate electrodeposition provides a programmable method for the spatiotemporally controllable assembly of cell populations for cell-based biosensing and for studying cell-cell signaling.

Example 3 demonstrates entrapment of E. coli cells within the calcium alginate hydrogel and subsequent liberation of the E. coli cells.

In another embodiment the invention provides hydrogels containing cells or a cell population and methods of forming the same. In one embodiment the hydrogel is a calcium alginate hydrogel.

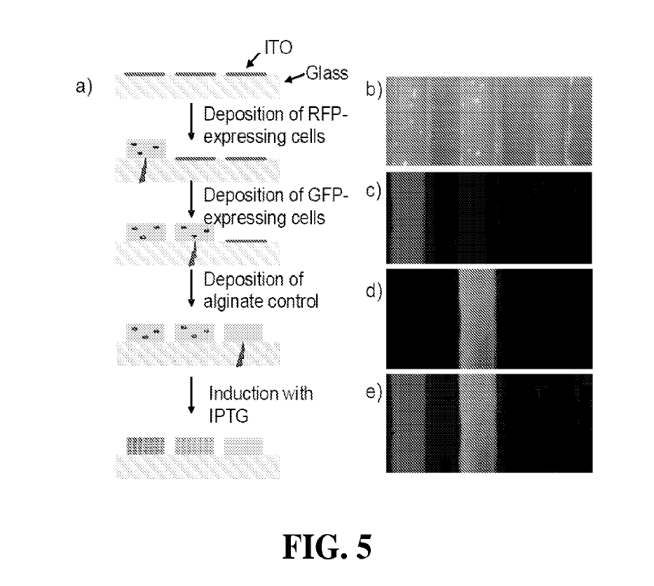

Electroaddressing by electrodeposition provides a simple means to perform multiplexed cell-based biosensing. To illustrate this capability, the experiment described as Example 4 below was performed, and is outlined in FIG. 5a. In this example two different populations of E. coli (RFP-expressing and GFP-expressing) were electrodeposited and entrapped at separate electrode addresses of the patterned ITO-coated slide in FIG. 5b.

The two images in FIGS. 5c and 5d are fluorescence photomicrographs using individual red and green filters while the image in FIG. 5e is a composite image. These images indicate that RFP-expressing cells are selectively confined on the left electrode address while GFP expressing cells are selectively confined on the middle address. No fluorescence is visible on the right-most, control electrode. This result indicates that there is little cross talk between the three electrodes. Specifically, the cells are electrodeposited at their specific address and did not substantially migrate to other addresses during the 18 hour experiment.

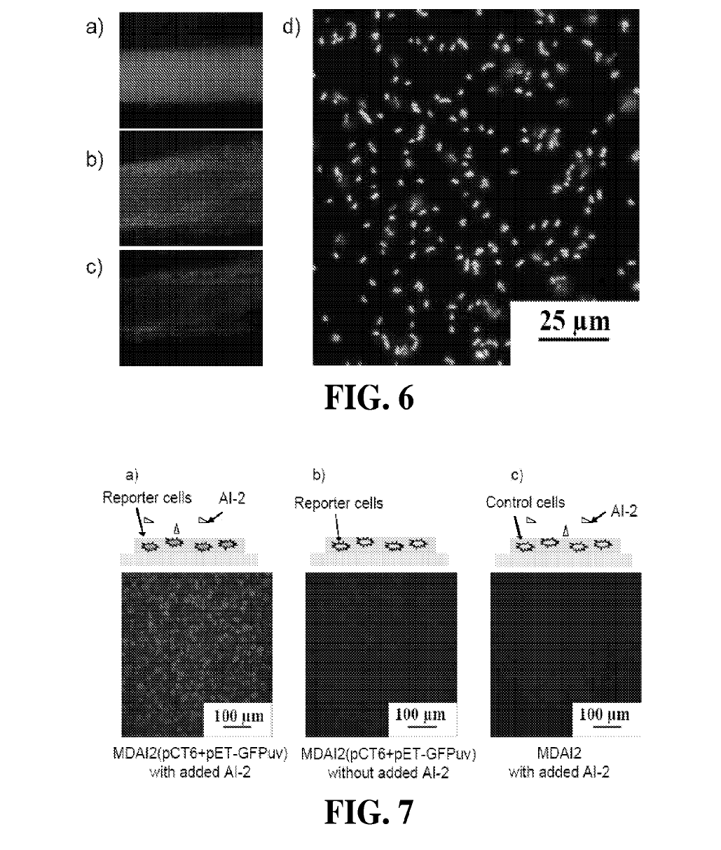

One advantage of using (bio)polymers that can undergo a reversible sol-gel transition is that the entrapped cells can be liberated from the matrix in response to external stimuli. In the case of calcium alginate, the hydrogel network can be disrupted by the addition of chemicals that preferentially bind calcium. To demonstrate this ability, recombinant E. coli was co-deposited onto a patterned ITO electrode (10×2 mm) and these cells were induced with IPTG to express GFP. After overnight incubation, the slide was examined and the photomicrograph of FIG. 6a shows strong fluorescence at this electrode address. A method of cell liberation is exemplified below in Example 5.

Additionally it was found by testing with bacterial quorum sensing (described in Example 6 below) that the alginate entrapped cells communicate with a cell population outside the alginate network.

This system is mediated by small signaling molecules known as autoinducers that are synthesized and secreted by bacteria, and later “sensed” by neighboring cells of the same or different species depending on the particular niche. Quorum sensing leads to changes in gene expression and cell phenotype, with a transition from single cell behavior to coordinated multicellular behavior. For instance, autoinducers have been implicated in controlling the virulence of the pathogenic E. coli O157:H7. In Example 6 below, MDAI2 E. coli which is unable to synthesize autoinducer 2 (AI-2) due to deletion of its terminal synthase gene, luxS was examined. Additionally a novel reporter strain, MDAI2 (pCT6+pET-GFPuv), which was engineered to respond to AI-2 by expressing GFP, was utilized. The results in FIG. 7 indicate that MDAI2 (pCT6+pET-GFPuv) is a useful reporter cell line that can detect externally added AI-2 and respond by expressing GFP.

Therefore in one embodiment the invention provides a method of cell-based biosensing by addition of a signaling molecule to a hydrogel as described herein where cells entrapped within the hydrogel detect the signaling molecule and respond to its presence. In another embodiment the hydrogel is a calcium alginate hydrogel.

The present invention provides methods for electrodeposition of calcium alginate hydrogel films in response to an anodic signal (i.e., a pH decrease) that triggers a localized release of calcium. Electrodeposition is achieved under sufficiently mild conditions that bacterial cells can be entrapped without destroying viability. The entrapped cells were observed to grow and respond to their environment (i.e., they could be induced). In addition, because gel formation is reversible, the entrapped cells can be liberated from the gels by the use of agents (i.e., citrate) that outcompete alginate for calcium binding.

The present invention therefore provides a reagentless method to electroaddress and entrap cells within a benign hydrogel matrix. Calcium alginate hydrogels are routinely used for microbiological cultivation and are often considered for tissue engineering scaffolds (M. Rinaudo, Polym. Int. 2008, 57, 397; J. M. Dang, K. W. Leong, Adv. Drug Deliv. Rev. 2006, 58, 487). The present invention provides a simple, rapid and benign method for the programmable electroaddressing of cell populations. The methods described herein have broad applications for cell based biosensing in array or microfluidic formats.

Therefore in one embodiment the invention provides a method for generating an assembly of cell populations, a microfluidic system and/or an assay comprising a hydrogel as described herein, containing one or more cell populations. In another embodiment the hydrogel is a calcium alginate hydrogel.

As shown herein, entrapped cells can “communicate” with external cell populations. Co-deposition of cells with calcium alginate or another electroaddressable polymer provides a convenient means to spatially segregate one population of cells (e.g., reporter cells) while allowing communication with co-cultured populations through diffusible signaling molecules. Thus, calcium alginate electrodeposition may provide a convenient experimental method for studying cell-cell signaling.

Thin film fabrication methods that allow complex biological systems to be integrated and probed on-chip further extend the power of microelectronics by enabling its application to biology and medicine. Ideally, the materials and methods for bio-device integration will enlist convenient electrical signals while accommodating the labile nature of biology.

Therefore in one embodiment the invention provides a hydrogel with stimuli-responsive polymer integrated therein. In one embodiment at least one stimuli-responsive polymer is a polysaccharide. The stimuli-responsive polymer may allow in-film bioprocessing of an entrapped a cell population. The invention provides an electroaddressable polymer blend for the in-film expansion of a cell population and probing of the cells' surface proteins by the electrophoretic migration of immunoreagents into the film. Such stimuli-responsive polymers provide simple, rapid and benign means to electroaddress films for in-film bioprocessing. Stimuli-responsive biological polymers form hydrogels in response to mild stimuli. These hydrogel networks can be reversibly formed/broken.

Stimuli-responsive polymers useful in hydrogels of the invention include, but are not limited to polymers subject to stimuli including, but not limited to, electroaddressability, electrical signals, temperature sensitivity, and pH sensitivity. Exemplary electroaddressable polymers include, but are not limited to, alginate, calcium alginate and chitosan. Temperature sensitive polymers include, but are not limited to, agarose and gelatin. In one embodiment the invention provides a hydrogel with at least two stimuli-responsive polymers, where the first stimuli-responsive polymer is an electroaddressable polymer and the first stimuli-responsive polymer is co-deposited with at least a second stimuli-responsive polymer.

The hydrogels of the invention are therefore useful in both cell-based and protein-based operations. However, because antibodies do not diffuse or migrate into alginate-rich gels, further experimentation was required to determine how to perform immunoanalysis of cells entrapped in an alginate gel. In order to successfully analyze the cells, antibody-based immunoreagents must be capable of penetrating into the hydrogel network to access the cells.

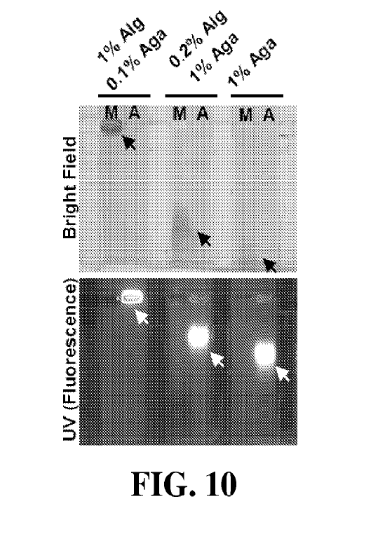

Alginate suppresses protein electrophoresis, as is shown by the experiment described in Example 7 and the results in FIG. 10. FIG. 10 demonstrates that no migration of the protein markers and the labeled antibody were observed in the alginate-rich gel at the left in FIG. 10, substantial protein migration was observed in the middle gel (LM-agarose gel (1%) containing a small amount of alginate (0.2%)), and rapid protein migration was observed in the LM-agarose gel at the right.

Two independently responsive polysaccharides were investigated to enable both electrodeposition (conferred by alginate) and electrophoresis (enabled by agarose). Electrodeposition was examined using both in situ and ex situ electrochemical quartz crystal microbalance (EQCM) measurements, as described in Example 8.

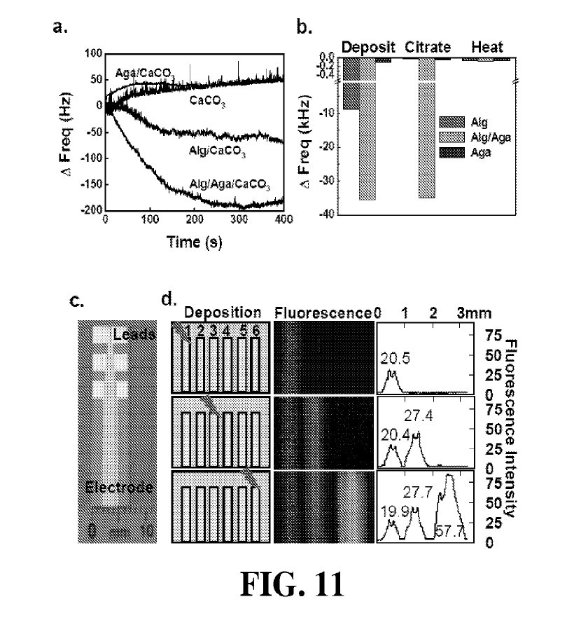

In situ EQCM results from several solutions are shown in FIG. 11a. For solutions lacking alginate, little change in resonant frequency is observed which is consistent with the expectation that neither CaCO₃ nor agarose can electrodeposit. The resonant frequency for the crystal immersed in the solution containing alginate and CaCO₃ decreased monotonically with time indicating the accumulation of mass on the anode (the 60 Hz decrease in resonant frequency corresponds to 84 ng). This result is in agreement with the visual observation that a thin hydrogel film is formed on the electrode surface. FIG. 11a shows an even larger decrease in resonant frequency (≈200 Hz corresponding to 280 ng) for deposition from a blend of alginate, LM-agarose and CaCO₃. Visually, a thicker film was observed to electrodeposit when LM-agarose was added to the alginate solution.

The in situ results demonstrate that deposition occurs from solutions containing alginate or a blend of alginate and LM-agarose, while no deposition is evident for LM-agarose. Accordingly alginate is shown to allow for the co-deposition of LM-agarose. Presumably the agarose chains are entrapped within the electrodeposited alginate network.

Ex situ EQCM results are shown in FIG. 11b. Deposition is seen to occur from a solution of alginate or a blend of alginate and LM-agarose, but not from a solution of LM-agarose. Accordingly, alginate is shown to electrodeposit in the presence or absence of LM-agarose. Further, the difference in resonant frequency indicates that more mass is deposited from the polysaccharide blend than for the alginate solution (49.8 vs. 12.4 μg).

The crystals formed with films of 1) alginate, 2) LM-agarose, or 3) a blend of alginate and LM-agarose, as described above were immersed in a solution containing sodium citrate to disrupt the calcium alginate network.

The ex situ EQCM measurement for the crystal with deposited alginate shows that the resonant frequency returns to the initial value (prior to deposition) which is consistent with the visual observation that calcium alginate films dissolve in sodium citrate. In contrast, both visual observation and the EQCM measurements show that the deposited film obtained from the alginate-agarose blend does not dissolve in citrate. Sodium citrate was seen to solubilize alginate film, but not alginate-agarose films.

Additionally, the crystals formed with films of 1) alginate, 2) LM-agarose, or 3) a blend of alginate and LM-agarose, as described above were incubated in hot water. Visually, the alginate-agarose film was observed to dissolve and this observation is consistent with the ex situ EQCM measurements. Hot water treatment was seen to solubilize alginate-agarose film.

The EQCM results of Example 8 demonstrate that alginate and LM-agarose can be co-deposited and that the electrodeposited blend forms a thermally-responsive network upon cooling, where agarose confers thermal-responsiveness to the film.

The electrodeposition methods of the present invention provide simple and rapid methods for the spatiotemporally controlled assembly of hydrogel films. Example 9 demonstrates such characteristics by sequential electrodeposition onto different electrode addresses of the chip in FIG. 11c, as described in Example 9, below.

The fluorescence photomicrographs and associated image analysis of the chip are shown in FIG. 11d. It can be seen that electrodeposition is achieved with high spatial and temporal control. The addition of LM-agarose to the blend led to increases in both fluorescence intensity and width of the deposited film which suggests that thicker films are deposited from the alginate-agarose blend. Profilometry measurements of films from various experiments indicate that the thickness of the dried films ranged from 0.5 to 2.5 μm with a consistent trend that thicker films were deposited from the blend. Furthermore, FIG. 11d shows that the average image intensity of a deposited film is not altered by subsequent deposition steps indicating that each deposition step can be performed independently without disruption of previously-deposited films.

Therefore, in one embodiment the invention provides a method of forming a calcium alginate hydrogel on a substrate, comprising co-depositing a source of calcium ions, a source of alginate and a source of a stimuli-responsive polymer in the presence of an electrochemically charged anode under conditions such that a calcium alginate gel is electrochemically deposited on the substrate in the location of the anode and the polymer is contained within the calcium alginate gel. In a specific embodiment the polymer is agarose.

In one embodiment, the invention provides a calcium alginate hydrogel with stimuli-responsive polymer chains entrapped therein. In a specific embodiment the invention provides an alginate-agarose hydrogel.

In another embodiment the invention provides a method of forming a hydrogel on a substrate, comprising co-depositing at least two stimuli-responsive polymers, wherein at least one of the stimuli-responsive polymers is an electroaddressable polymer and wherein a second stimuli-responsive polymer is responsive to at least one of: thermal changes, pH changes, and electrical signals, under conditions such that a hydrogel comprising the at least two stimuli-responsive polymers is electrochemically deposited on the substrate.

In still another embodiment the invention provides a hydrogel comprising at least two stimuli-responsive polymers, wherein at least one of the stimuli-responsive polymers is an electroaddressable polymer and wherein a second stimuli-responsive polymer is responsive to at least one of: thermal changes, pH changes, and electrical signals.

In another embodiment the invention provides a method of forming a hydrogel as described herein with one or more cell populations within the film, and wherein the film permits in-film bioprocessing of the cell population.

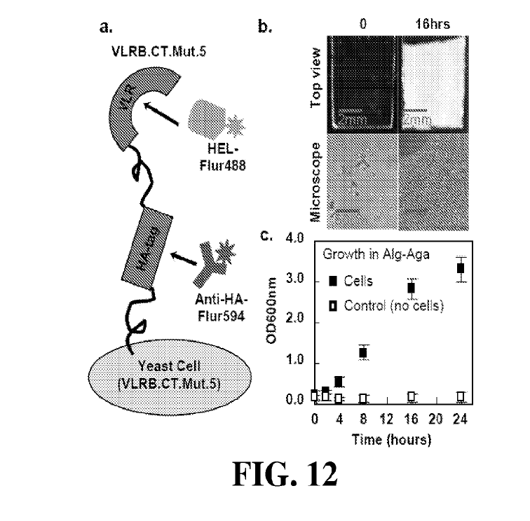

A model biological system of the present invention is a yeast strain that has been engineered to display on the cell surface a variable lymphocyte receptor (VLR) from sea lamprey. VLRs are unique proteins that serve as antigen receptors in the adaptive immune system of jawless vertebrates. Since VLRs are assembled by recombinatorial DNA rearrangements and can recognize and bind any nominal antigen, they are functionally analogous to mammalian antibodies. However, VLRs are structurally different from antibodies, consisting of leucine-rich repeats instead of immunoglobulins, and this difference has sparked considerable fundamental and technological interest. Recently, VLRs that bind the model protein hen egg lysozyme (HEL) were selected from libraries constructed in a novel yeast surface-display vector, where the VLRs were N-terminally fused to a yeast surface anchor, separated by a spacer encoding a hemagglutinin (HA) tag. FIG. 12a provides an illustration of a yeast (VLRB.CT.Mut5) model showing the HEL-specific VLR with HA displayed on the cell surface. The antigen binding properties of several clones were rationally engineered by directed evolution for enhanced affinity and the clone with the most improved HEL binding ability (improved from K_{D}=155 nM to K_{D}=119 pM) was designated clone VLRB.CT.Mut5.

This biological system allows inducible expression of monoclonal VLRs on the yeast surface, and the displayed protein can be detected by binding of both the HEL antigen (≈15 kDa) and the much larger immunoreagent anti-hemagglutinin (anti-HA; ≈150 kDa).

Example 10 demonstrates that the yeast can proliferate in electrodeposited alginate-agarose films. The upper images in FIG. 12b are photographs of the film immediately following electrodeposition and after overnight incubation at 30° C. As evident from these photographs, the film became considerably more opaque during cultivation due to growth of the yeast. The lower images in FIG. 12b are photomicrographs that further indicate extensive growth of the yeast during the overnight incubation.

A growth curve for yeast in the electrodeposited films was obtained by generating several films on ITO-coated glass slides as described above and harvesting individual films after specific incubation times. The yeast entrapped in the harvested films were liberated using citrate (25 mM) and a commercial chaotropic solution known to solubilize LM-agarose gels. After releasing the yeast from the films, the cells were centrifuged, washed, re-suspended in water and the optical density of the resulting suspension was measured at 600 nm. The results in FIG. 12c show substantial increases in optical density for the films “inoculated” with yeast, while films deposited without yeast show no change in optical density. The doubling time for the entrapped yeast was estimated to be 3 hr which is comparable to the doubling time of suspension cultured yeast. Thus, yeast co-deposited and entrapped within the alginate-agarose blend can proliferate in these films.

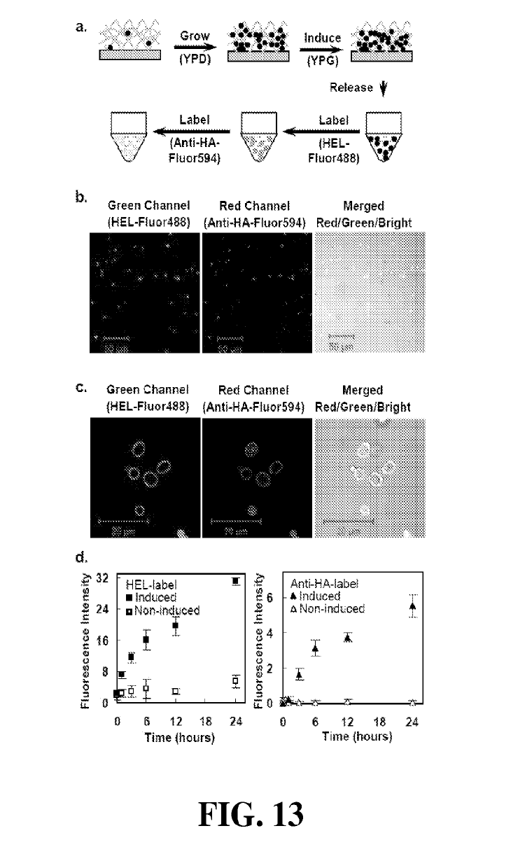

Example 11 and FIG. 13a demonstrate that film cultivated yeast (VLRB.CT.Mut5) can be induced to express and display VLR protein. Specifically, yeast were co-deposited in an alginate-agarose film onto slides, the slides were exposed sequentially to YPD growth medium and then YPG induction medium. The cells were released from the films with citrate and chaotropic solution to analyze VLR expression. The released cells were first incubated with the labeled HEL antigen, washed and then incubated with labeled anti-HA antibody. The images from confocal laser scanning microscopy are shown in FIG. 13b. These images indicate considerable green fluorescence (left image; indicating binding of fluorescently-labeled HEL antigen), red fluorescence (center image; indicating binding of anti-HA antibody) and co-localization of the fluorescence (right image; suggesting the antigen and anti-HA binding sites are on the same yeast). Little fluorescence was observed for un-induced control cells. Thus, the images in FIG. 13b demonstrate that film-cultivated yeast can be induced to express VLR. Confocal images at higher magnification are shown in FIG. 13c. These images also show co-localization of the green and red fluorescence and further suggest that the VLR is displayed on the yeast surface.

FIG. 13d provides the induction curves for film-cultivated yeast, as described in Example 11, which indicate a steady and simultaneous increase in both green and red fluorescence consistent with the expression of VLR. FIG. 13d also shows minimal fluorescence for the non-induced controls.

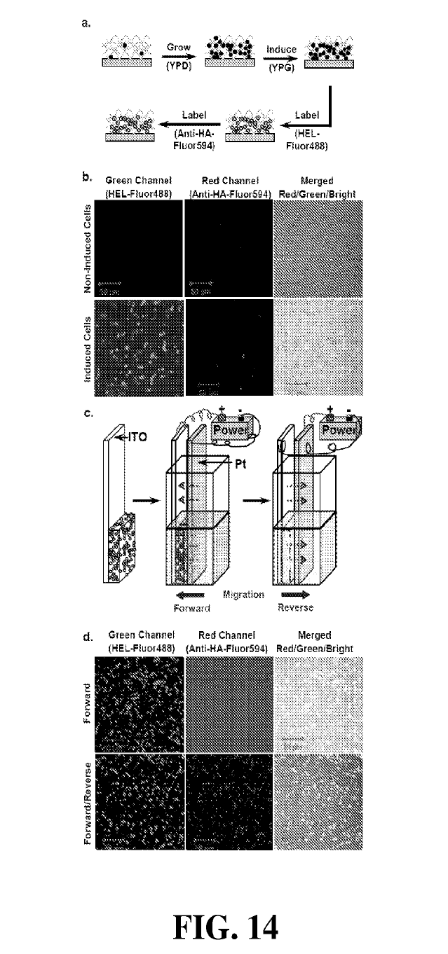

Example 12 and FIG. 14a demonstrate in-film immunoanalysis of the entrapped VLRB.CT.Mut5 yeast with fluorescently-labeled HEL antigen and anti-HA antibody. The micrograph in FIG. 14b for the non-induced control shows no fluorescence with either the green filter (labeled-HEL antigen) or red filter (labeled anti-HA) consistent with the absence of surface displayed VLR. The induced culture in FIG. 14b shows considerable fluorescence with the green filter indicating that the HEL antigen can diffuse into the alginate-agarose film and bind to the VLR. However, little fluorescence was observed using the red filter suggesting that the larger and more acidic anti-HA antibody has limited ability to diffuse into the hydrogel network.

It was also found that yeast co-deposited with alginate and without agarose can proliferate and express VLR; however the alginate-entrapped cells could not be probed with the anti-HA immunoreagent.

Therefore in one embodiment the invention provides a method of in-film bioprocessing, comprising: electrodepositing an alginate-agarose hydrogel comprising at least a first cell population, wherein the first cell population comprises cells capable of expressing a surface expressed protein; proliferating the cells in the alginate-agarose hydrogel; inducing expression of the surface expressed protein; adding an immunoreagent; and determining occurrence of binding of the immunoreagent and the surface expressed protein to form an immunocomplex.

As further described in Example 12, electrophoresis was examined for its ability to enable the anti-HA immunoreagent to penetrate the film and access the entrapped yeast. The green fluorescence image at the top left in FIG. 14d is consistent with the binding of the HEL antigen to cells. The middle image at the top in FIG. 14d shows red fluorescence is observed throughout the field indicating that the labeled anti-HA antibody has penetrated into the film due to the applied electric field. This observation is consistent with FIG. 10 which shows that proteins can readily migrate through a gel prepared from alginate and LM-agarose.

A second sample was prepared by performing two electrophoresis steps. The fluorescence images at the bottom in FIG. 14d show red fluorescence is observed in localized regions and these regions are co-localized with the green fluorescence obtained from the labeled HEL antigen.

Therefore in one embodiment the invention provides a method of in-film bioprocessing, comprising: electrodepositing an alginate-agarose hydrogel comprising at least a first cell population, wherein the first cell population comprises cells capable of expressing a surface expressed protein; proliferating the cells in the alginate-agarose hydrogel; inducing expression of the surface expressed protein; adding an immunoreagent under electrophoresis; and determining occurrence of binding of the immunoreagent and the surface expressed protein to form an immunocomplex.

The term “immunoreagent” as used herein refers to reagents for use in in-film bioprocessing such as immunoanalysis, wherein the reagents bind to the cells entrapped within a hydrogel. Immunoreagents may include, but are not limited to antigens, antibodies, and active fragments or portions thereof. The immunoreagents may be further tagged or labeled or otherwise conjugated to an agent useful in in-film bioprocessing. In one embodiment the immunoreagents include fluorescently labeled HEL antigen and fluorescently labeled anti-HA antibody.

The invention therefore provides that low levels of alginate allow for the co-deposition of other, stimuli-responsive biopolymers, such as agarose, which extend the utility of electrodeposition. First, it enables the co-deposition of polymers that can form films in response to additional stimuli rather than simply a pH gradient. Second, it extends deposition to neutral polymers that allow access to a broader range of biotechnological procedures (e.g., electrophoresis). From a device perspective, electrodeposition with stimuli-responsive biopolymers is potentially significant because: it enlists convenient electrical signals for programmable assembly (Yi, H. M., et al., Biomacromolecules 2005, 6, 2881; Park, J. J., et al., Lab Chip 2006, 6, 1315; Luo, X. L., et al., Lab on a Chip 2008, 8, 420). From a biology perspective, electrodeposition provides a rapid, reagentless and biocompatible means to electroaddress biological materials (Shi, X. W. et al. Advanced Functional Materials, 2009, 19, 13, 2074; Shi, X. W., et al. Advanced Materials, 2009, 21, 984). Thus, electrodeposition with stimuli-responsive biopolymers may enable in-film bioprocessing for applications that include: evaluating biopsy samples for enhanced diagnosis and personalized medicine; providing experimental models of drug metabolism to facilitate discovery and testing; and understanding spatiotemporally controlled developmental cues for tissue engineering.

The advantages and features of the invention are further illustrated with reference to the following examples, which are not to be construed as in any way limiting the scope of the invention but rather as illustrative of one embodiment of the invention in a specific application thereof.

Various materials used in the examples below were obtained as follows: the following materials were purchased from Sigma-Aldrich: sodium alginate from Macrocystis Pyrifera (medium viscosity), calcium carbonate power (10 μm), CaCl₂ pellets, phosphate buffered saline (PBS, pH 7.4), isopropyl β-D-1-thiogalactopyranoside (IPTG), FITC-labeled microparticles based on melamine resin (1 μm), indium tin oxide (ITO)-coated glass slides (surface resistivity 8-12 Ω/sq) sodium alginate from brown algae (medium viscosity), and hen egg lysozyme (HEL; 14.7 kDa).

Luria-Bertani (LB) medium was purchased from Acros. Silicon wafers were patterned with gold using standard photolithographic methods as previously described (L.-Q. Wu, et al., Langmuir, 2003, 19, 519).

Additional chemicals were purchased; low melting point agarose (LM-agarose; Promega), tris(hydroxymethyl)aminomethane (Tris; Fischer), NHS-fluorescein (Pirece), red-fluorescent Alexa Fluor 594 anti-HA antibody (Invitrogen) and protein markers (EZ-Run Pre-Stained RecProtein Ladder; Fisher Scientific). The commercial chaotropic solution (4.5 M isothiocyanate, 0.5 M acetate, pH 5) used was the “membrane binding solution” from Wizard SV Gel and PCR Clean-up System (Promega).

The YPD (yeast, peptone, dextrose) growth medium contains Bacto yeast extract (10 g/L), Bacto proteose peptone (20 g/L), dextrose (i.e., glucose; 20 g/L) and the antibiotic geneticin (G418; 100 μg/ml). The YPG (yeast, peptone, galactose) induction medium contains Bacto yeast extract (10 g/L), Bacto proteose peptone (20 g/L), galactose (20 g/L) and the antibiotic geneticin (G418; 100 μg/ml). Unless otherwise noted, yeast were codeposited from a warm suspension containing yeast (optical density 0.2), alginate (0.2%), LM-agarose (1.0%) and CaCO₃ (0.25%) onto an ITO-coated glass microscope slide using a constant current density (2 A/m²) for 1 min.

Several standard experimental methods were used in this study. Chips were prepared using conventional microfabrication methods to pattern gold onto silicon wafers. Hen egg lysozyme (HEL) was fluorescently labeled with NHS-fluorescein using a standard labeling kit and instructions provided by the supplier (Pierce NHS-Fluorescein Antibody Labeling Kit). Image analysis of the fluorescence photomicrographs of the electrodeposited microparticles on the patterned chip was performed using Image J software (available at hyper text transfer protocol internet address: rsb.info.nih.gov/ij/). Electrophoresis was performed using standard electrophoresis buffer composed of 25 mM Tris and 250 mM Glycine (pH 8.3). Changes in the resonant frequency measured with the quartz crystal microbalance were converted into mass changes using the Sauerbrey equation.

The following instruments were used in this study: spectrophotometer (Thermo Scientific, Evolution60), electrochemical quartz crystal microbalance (EQCM; CH Instruments, Electrochemical Analyzer), fluorescence plate reader (SpectraMax M2 Microplate Readers), confocal laser scanning microscope (Zeiss, LSM510 meta), fluorescence microscope (Leica MZ FLIII, with GFP2 filter), and power supply (Keithley, 2400 sourcemeter).

### Example 1

Calcium Alginate Electrodeposition

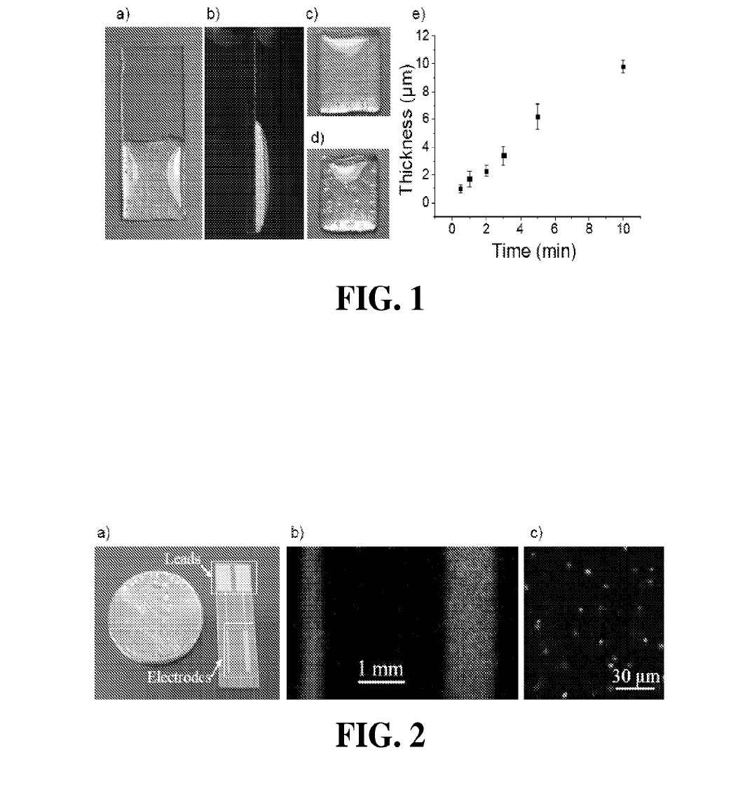

In this experiment CaCO₃ powder (10 μm particles; 0.25%) was blended into a sodium alginate (1.0%) solution and sonicated for 10 min. An ITO coated glass slide (2.5×1.0 cm) was partially immersed into this deposition solution and an anodic voltage was applied to achieve a current density of 3 A/m2 (typical voltage is about 3.5 V) for 5 min (a platinum film served as the cathode). These conditions yield a thick deposit of calcium alginate. After electrodeposition, the ITO slide was removed from the solution and rinsed briefly with NaCl (0.1 M) solution, and then disconnected from the power supply. The photograph in FIG. 1a shows the deposited film is relatively opaque (due to entrapped CaCO₃ particles) and contains several trapped bubbles (due to the generation of CO₂ and O₂). A side view of this moist film is shown in FIG. 1b from which the thickness can be estimated to be about 1 mm. When electrodeposited films are sufficiently thick, they can be peeled from the substrate as illustrated in FIG. 1c.

To provide evidence that the film's opacity is due to entrapped CaCO₃ particles the film was immersed in acid (0.1 M HCl) for 10 minutes. FIG. 1d shows that the acid-treated film is transparent due to the solubilization of the CaCO₃.

To demonstrate that the electrodeposition of calcium alginate can be controlled, films were electrodeposited for various times. After deposition, the films were air dried at room temperature for 24 hr and the film thickness was measured by profilometry. FIG. 1e shows an increase in film thickness with deposition time. These results demonstrate that calcium alginate gels can be controllably electrodeposited at the anode.

### Example 2

Spatial Selectivity of Calcium Alginate Electrodeposition

The chip shown in FIG. 2a, which possesses two patterned gold electrodes (0.25 mm and 1 mm), was used to examine the spatial selectivity of calcium alginate electrodeposition. To facilitate visualization fluorescently labeled microparticles (1 μm; 0.1%) were added into alginate-CaCO₃ suspension (1% alginate; 0.25% CaCO₃). Deposition was performed by connecting both electrodes to the power supply and applying an anodic voltage at 1 A/m2 for 2 min.

The fluorescence photomicrograph in FIG. 2b shows that fluorescence is observed on both electrodes and that fluorescence is spatially confined in the lateral dimensions to the electrode surfaces. The graininess of the fluorescence in FIG. 2b suggests that the microparticles are entrapped as individual particles. To support the conclusion of individual particle entrapment, the internal region of the deposited film was imaged using confocal fluorescence microscopy. FIG. 2c shows a fluorescence image from the middle depth of the alginate film and indicates that the microparticles are dispersed and entrapped throughout the gel network.

The results in FIG. 2 demonstrate that calcium alginate electrodeposition is spatially selective in the lateral dimension.

Initial electrodeposition studies were performed using either ITO-coated slides or patterned silicon chips as anodes, and a platinum film as the cathode. The deposition solution was prepared by suspending CaCO₃ powder (10 μm particles; 0.25%) into a sodium alginate (1.0%) and sonicating for 10 min. For deposition with ITO-coated glass slides, the slides were partially immersed in the deposition solution and an anodic voltage was applied to achieve a current density of 3 A/m² (the deposition time was varied). To measure thickness of the electrodeposited calcium alginate, the ITO-coated slide was dried in air for 24 hr and measured using a profilometer (Alpha-step 500 Surface Profiler, TENCOR Instruments).

To examine the spatial selectivity for electrodeposition, FITC-labeled microparticles (1 μm particles, 0.1%) were blended into the deposition solution and deposition was performed using a chip with patterned gold electrodes. For deposition, the chip was partially immersed in the deposition solution and an anodic voltage was applied for 2 min to achieve 1 A/m² (a lower current density was used to prevent destruction of the gold electrodes). The microparticle-containing films deposited on the patterned electrodes were imaged using a Leica fluorescence microscope (MZFL III) connected with a digital camera (spot 32, Diagnostic Instrument).

### Example 3

Entrapment, Growth, Induction and Liberation of E. Coli Cells

The potential for co-depositing and entrapping E. coli cells within electrodeposited calcium alginate gels was examined. The cells for inoculation were initially cultured in LB medium to an OD₆₀₀ of 1.0 and then diluted 10-fold in a suspension of alginate and CaCO₃ (final concentrations; 0.9% alginate and 0.23% CaCO₃) that had been previously autoclaved. An ITO-coated slide that had been sterilized with ethanol was partially immersed in this suspension and an anodic voltage was applied to a current density of 3 A/m² for 2 min.

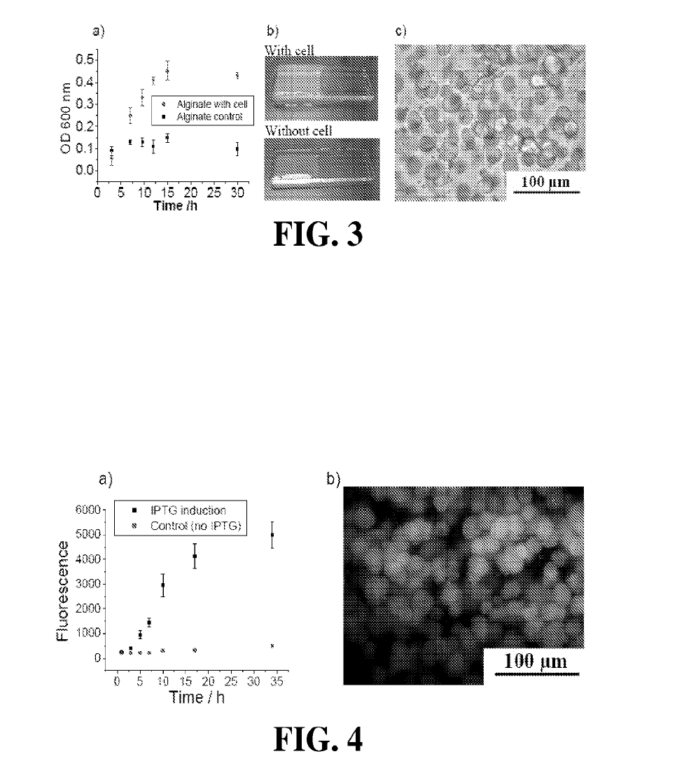

After electrodeposition, the resulting gel was washed with 1.0% NaCl and hardened by immersion in 1.0% CaCl₂ solution for 30 min at 4° C. The slide with the entrapped cells was then incubated with 2 ml LB medium at 37° C., and the optical density was intermittently measured. The growth curve in FIG. 3a shows a steady increase in optical density for the inoculated gels, while a control film electrodeposited without cells shows no change in optical density. The photographs of the inoculated and control alginate films in FIG. 3b show an obvious difference in optical density between these hydrogel films.

The bright field image in FIG. 3c was obtained after incubating the cells for 7 hours in the alginate gel. This image indicates that cell growth is accompanied by the appearance of 20-30 μm colonies. Presumably the colonies are the result of cell division that is spatially confined to specific regions of the alginate gels. No colonies were observed in the inoculum or immediately after co-depositing cells within the alginate gel (images not shown). This observation would suggest that the bacterial cells are entrapped and unable to freely move through the calcium alginate network (i.e., daughter cells are retained near the mother). The results in FIG. 3 indicate that E. coli can be co-deposited and entrapped within calcium alginate gels, and that co-deposited cells remain viable and are able to grow.

Next it was examined whether co-deposited E. coli cells entrapped within an alginate gel could be induced to express a foreign protein in response to an externally-added inducer. Recombinant cells that express green fluorescent protein (GFP) in response to IPTG induction were used.

Using methods described above, the cells were co-deposited with alginate and CaCO₃, hardened with CaCl₂, and then incubated in LB medium at 37° C. for 2 h. After this initial incubation, IPTG (1 mM final concentration) was added and the fluorescence of the gel was monitored. FIG. 4a shows that 3h after adding IPTG, the fluorescence began to increase and a nearly linear increase in fluorescence was observed until 17 h. The control cells that had been co-deposited but not induced by IPTG showed little change in fluorescence during incubation. At the end of the experiment the induced cells were imaged using a fluorescence photomicroscope. The image in FIG. 4b shows that the fluorescence for the induced cells appears to be spatially confined to small regions (i.e., colonies) within the alginate gel. This spatial confinement of induced cells within the alginate network is consistent with observations in FIG. 3c. The results in FIG. 4 demonstrate that entrapped cells can respond to their environment (i.e., they can be induced).

In this example, and as described elsewhere in this application, various methods were used to examine the entrapped cells: optical density was measured using a spectrophotometer (DU640 Beckman); fluorescence was quantified using a fluorescence microplate reader for alginate films that had been peeled from the slides (SpectraMax5, Molecular Devices); bright field images were obtained using a confocal microscope (1×81-DSU, Olympus); fluorescence images were obtained using a fluorescence microscope (BX-60, Olympus or MZFL III Leica) and a scanning confocal laser microscope (Zeiss LSM 510 with an Ar laser at 488 nm).

### Example 4

Demonstration of Electroaddressing by Calcium Alginate Electrodeposition

RFP-expressing E. coli was co-deposited by immersing the patterned slide in the cell-alginate-CaCO₃ suspension and biasing the left-most electrode (3 A/m² for 2 min). After washing with NaCl, GFP-expressing E. coli was co-deposited by immersing the slide in a second cell-alginate-CaCO₃ suspension and biasing the middle electrode. After rinsing with NaCl, calcium alginate (without cells) was electrodeposited at the right-most, control electrode. This slide was incubated in LB medium for 2 hours after which the IPTG inducer was added and then the slide was further incubated overnight for an additional 16 hours.

Strains that express green fluorescent protein (GFP) and red fluorescent protein (RFP) in response to IPTG induction were described elsewhere (X. Yang, et al., Langmuir 2009, 25, 338). These strains were cultured in LB medium containing 50 μg/mL kanamycin and 34 μg/mL chloramphenicol for use in the present Example.

### Example 5

Liberation of Entrapped Cells

To liberate the alginate-entrapped cells, the slide was immersed in PBS buffer (100 mM; pH 7.4) containing sodium citrate (500 mM) and gentle shaking was applied. As illustrated in FIG. 6b, the alginate gel began dissolving within 2 min and dissolution was nearly complete in 5 min (FIG. 6c). FIG. 6d shows that the liberated E. coli appear as individual cells (and not multicellular colonies). These results demonstrate that the alginate-entrapped cells can be liberated intact by the addition of sodium citrate that can bind calcium.

### Example 6

Communication with External Cell Population

MDAI2 (pCT6+pET-GFPuv) reporter cells were electrodeposited in the calcium alginate gel. These entrapped cells were then incubated in 2 ml LB medium containing 200 μA of a solution obtained from the in vitro-synthesis of AI-2. After incubation at 26° C. for 16 hr, the entrapped cells were imaged using fluorescence photomicroscopy. FIG. 7a shows considerable GFP fluorescence for these entrapped reporter cells which indicates that they are able to respond to the added AI-2. One control in FIG. 7b is entrapped MDAI2 (pCT6+pET-GFPuv) reporter cells incubated with LB that lacked AI-2 addition. As expected, no GFP fluorescence is observed in the image for this control. A second control in FIG. 7c is entrapped MDAI2 cells that are neither able to synthesize AI-2 nor respond to AI-2 by expressing GFP. As expected, incubation of these entrapped cells with AI-2 does not lead to the induction of GFP as evidenced from the image in FIG. 7c.

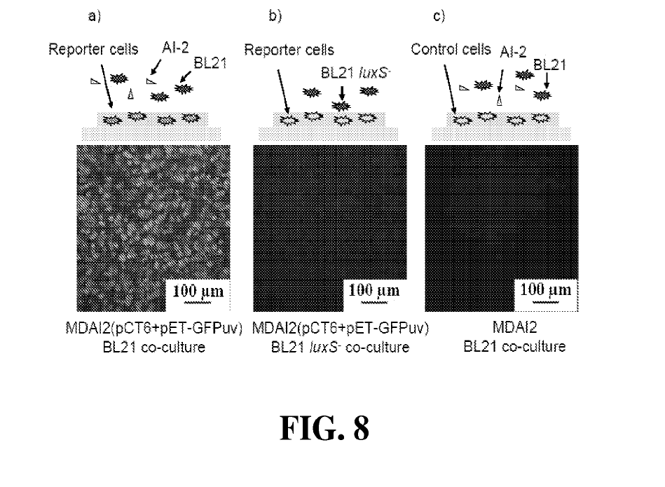

Furthermore, MDAI2 (pCT6+pET-GFPuv) reporter cells were electrodeposited in the calcium alginate gel. These entrapped cells were then incubated in 2 ml LB medium and a second culture, BL21 E. coli, was inoculated into the liquid phase (2 μA of BL210D₆₀₀=3.9). The liquid phase BL21 E. coli can produce and secrete AI-2 and should be able to signal to the entrapped reporter cells that can detect and respond to AI-2. These co-cultures were incubated at 26° C. for 16 hr. The fluorescence photomicrograph in FIG. 8a shows that the entrapped reporter cells became fluorescent in this co-culture. The first control in FIG. 8b is a co-culture of entrapped MDAI2 (pCT6+pET-GFPuv) reporter cells with a BL21 luxS knockout strain in the liquid phase. This BL21 luxS knockout strain is unable to synthesize AI-2 and thus should be unable to generate the quorum sensing signaling molecule to induce GFP expression in the entrapped reporter cells. As expected, the fluorescence photomicrograph in FIG. 8b shows no fluorescence for the entrapped reporter cells in this control. The second control in FIG. 8c is a co-culture of entrapped MDAI2 (unable to synthesize AI-2 or express GFP in response to AI-2) and liquid phase BL21 which can synthesize AI-2. As expected, FIG. 8c shows no fluorescence in this control. The results in FIGS. 7 and 8 indicate that the entrapped reporter cells are capable of reporting the presence of the AI-2 signaling molecule.

E. coli MDAI2 is a luxS knockout strain that is unable to synthesize AI-2. MDAI2 (pCT6+pET-GFPuv) is obtained by transforming MDAI2 with two plasmids to enable this strain to respond to exogenously-added AI-2 by expressing GFP. E. coli BL21 (Novagen) was used in co-culture experiments and this strain can produce endogenous AI-2. The luxS knockout mutant, E. coli BL21 luxS, is unable to produce AI-2. In vitro AI-2 was synthesized using a procedure described previously (A. F. Gonzalez Barrios, et al., J. Bacteria, 2006, 188, 305; R. Fernandes, et al., Metab. Eng., 2007, 9, 228). Briefly, 1 mM S-adenosylhomocysteine was reacted with the two purified enzymes His6-Pfs and His6-LuxS in 50 mM Tris-HCl (pH=7.8) at 37° C. for 4 hr. The conversion was estimated to be about 60% based on the quantification of free thiols by DTNB (5,5′-Dithiobis (2-nitrobenzoic acid).

### Example 7

Evaluation of Protein Electrophoresis in Gels

Three gels were poured from low melting agarose (LM-agarose), or a blend of LM-agarose plus sodium alginate. For each gel, protein markers (designated “M” in FIG. 10; 10 μg) were loaded in the left lane and a fluorescently-labeled antibody (designated “A” in FIG. 10; Alexa Fluor 594-labeled anti-hemagglutinin; 5 μg) was loaded in the right lane. After electrophoresis (1 V/mm for 30 min) in standard electrophoresis buffer (25 mM Tris, 250 mM Glycine; pH 8.3), the gels were imaged using bright field (upper panel) and fluorescence (lower panel).

### Example 8

Evaluation of Electrodeposition in Gels

For in situ EQCM experiments, the quartz crystals were immersed in polysaccharide-containing solutions that were dilute (to enable in situ measurement) and warm (37° C.; to ensure LM-agarose remained soluble). Solutions contained 0.006% CaCO₃ and alginate (0.02%); a blend of alginate (0.02%) plus LM-agarose (0.1%); or LM-agarose (0.1%).

Anodic deposition was initiated by applying a constant voltage (+2.5 V) to the working electrode that was patterned on the crystal and the change in resonant frequency of the crystal was monitored over time. In situ EQCM results from several solutions are shown in FIG. 11a.

Ex situ EQCM measurements were performed to demonstrate that agarose's thermally-responsive properties are retained upon co-deposition with alginate. Deposition was performed from warm polysaccharide solutions (37° C.; +2.5 V for 1 min). Solutions contained 0.25% CaCO₃ and alginate (0.2%); a blend of alginate (0.2%) plus LM-agarose (1.0%); or LM-agarose (1.0%).

After deposition the films were cooled (to allow the LM-agarose network to form) and dried at room temperature, and the resonant frequency was measured in air. The results are shown in FIG. 11b.

Next, the crystals with the deposited films were immersed in a solution containing sodium citrate (50 mM for 10 min) to evaluate disruption of the calcium alginate network.

Finally, the crystals were incubated in hot water (80° C. for 20 min) to evaluate disruption of the calcium alginate network.

### Example 9

Spatiotemporally Controlled Assembly of Hydrogel Films by Electrodeposition

Using the chip in FIG. 11c, films were sequentially electrodeposited onto different electrode addresses (each address is 250 μm wide gold line spaced 250 μm apart). The deposition sequence illustrated in FIG. 11d was: (i) deposit from alginate (0.2%) onto electrode #1, (ii) deposit from an alginate (0.2%) plus LM-agarose (0.5%) blend onto electrode #3; and (iii) deposit from an alginate (0.2%) plus LM-agarose (1.0%) blend onto electrode #6. Fluorescently-labeled microparticles and CaCO₃ (0.25%) were included in all deposition solutions and deposition at the individual electrodes was achieved by biasing the single electrode for 8 sec at a current density of 20 A/m² (typically the voltage did not exceed 2.5 V). After each deposition step, the chip was cooled to room temperature and imaged using a fluorescence microscope.

### Example 10

Co-Deposition and in Film Proliferation of Yeast Cells

In these experiments, the warm suspension containing yeast (optical density 0.2), alginate (0.2%), LM-agarose (1.0%) and CaCO₃ (0.25%) was electrodeposited (2 A/m² for 1 min) onto an ITO-coated glass microscope slide. After deposition, the films were rinsed with warm water (37° C.) and then briefly immersed in cold YPD growth medium (4° C.) to allow the agarose gels to form. The upper images in FIG. 12b are photographs of the film immediately following electrodeposition and after overnight incubation (16 hr) at 30° C.

A growth curve for yeast in the electrodeposited films was obtained by generating several films on ITO-coated glass slides as described above and harvesting individual films after specific incubation times. The yeast entrapped in the harvested films were liberated using citrate (25 mM) and a commercial chaotropic solution known to solubilize LM-agarose gels. After releasing the yeast from the films, the cells were centrifuged, washed, re-suspended in water and the optical density of the resulting suspension was measured at 600 nm. The results in FIG. 12c show substantial increases in optical density for the films “inoculated” with yeast, while films deposited without yeast show no change in optical density. The doubling time for the entrapped yeast was estimated to be 3 hr which is comparable to the doubling time of suspension cultured yeast.

### Example 11

In Film Induction of Protein Expression

Yeast cells were co-deposited in an alginate-agarose film on slides. The process is schematically illustrated in FIG. 13a. The slides were incubated for 3 hours in the YPD growth medium, and then transferred to the YPG induction medium for 16 hours.

To analyze VLR expression, the cells were released from the films with citrate and chaotropic solution, collected by centrifugation, washed and suspended in PBS buffer (10 mM phosphate; 150 mM NaCl; pH 7.4). To detect VLR expression, the released cells were first incubated with the labeled HEL antigen (100 nM in PBS for 1 hr), washed and then incubated with labeled anti-HA antibody (5 μg/ml in PBS for 1 hour). Confocal images of induced yeast are provided in FIG. 13b and at higher magnification in FIG. 13c.

A time-course for VLR induction was obtained by co-depositing yeast onto several ITO-coated slides and harvesting individual samples after specified times post-induction. For analysis, the yeast were released from the film, incubated simultaneously with labeled HEL antigen (100 nM) and labeled anti-HA (5 μg/ml) for 1 hr, washed and then measured using a fluorescence plate reader. The fluorescence results are provided in FIG. 13d.

### Example 12

In Film Immunoanalysis of Entrapped Cells

Yeast cells were co-deposited in an alginate-agarose film on slides. Bovine serum albumin (BSA; 1.0%) was included in the deposition solution to limit non-specific binding during the subsequent immunoanalysis. After deposition, the slides were incubated in YPD growth medium (3 hr) and then YPG induction medium (4 hours). After induction, the slides were rinsed and incubated with a Tris buffer (50 mM; pH 7.5) containing the fluorescently-labeled HEL antigen (100 nM for 1 hr), and then rinsed and incubated with fluorescently-labeled anti-HA antibody (5 μg/ml in Tris for 1 hour). The micrograph in FIG. 14b shows the resulting fluorescence.

Further, electrophoresis was examined for its ability to enable the anti-HA immunoreagent to penetrate the film and access the entrapped yeast. The yeast were electrodeposited, grown, induced and contacted with the labeled HEL antigen as described above. Next the slides were inserted into the electrophoresis device shown in FIG. 14c with a platinum foil serving as the counter electrode. Electrophoresis was performed by adding labeled anti-HA (5 μg/ml) to the electrophoresis buffer and applying an electric field of 1 V/mm for 20 minutes (ITO coated slide served as the positive electrode). The fluorescence images in FIG. 14d shows the resulting fluorescence.

A second sample was prepared by performing two electrophoresis steps. Initially an electric field of 1 V/mm was applied for 20 minutes (ITO coated slide served as the positive electrode) to drive the anti-HA into the film. After replacing the solution with protein-free electrophoresis buffer, a second electrophoresis step was performed at 1 V/mm for 30 minutes in the opposite direction (the ITO-coated slide served as the negative electrode) to remove unbound anti-HA from the film. The fluorescence images in FIG. 14d shows the resulting fluorescence.

### INDUSTRIAL APPLICABILITY

The methods of the invention are useful for the formation of hydrogels by electrodeposition. Formation by electrodeposition provides an advantage in that it permits electroaddressing of the electrodeposited components. The electrodeposition of the components of the hydrogels is reagentless and benign and is also useful in formation of hydrogels that contain cells. Within the hydrogels the cells remain viable, can proliferate, can respond to the environment and can be released from the hydrogel. The electrodeposition methods are also useful in co-deposition of two or more stimuli-responsive polymers, resulting in stable hydrogels with physical, chemical and/or biological properties tailored by the polymers selected.

The stable hydrogels described herein are usefully employed in a wide variety of applications, including as matricies for biotechnology, tissue engineering, and in biosensing, bioprocessing, microarray and microfluidic applications.

1. A method of forming a calcium alginate hydrogel on a substrate, comprising co-depositing a source of calcium ions and a source of alginate in the presence of an electrochemically charged anode under conditions such that a calcium alginate gel is electrochemically deposited on the substrate in the location of the anode.

2. The method of claim 1, wherein the source of calcium ions is CaCO₃.

3. The method of claim 1, wherein the source of alginate is sodium alginate.

4. The method of claim 1, further comprising co-depositing agarose, such that the agarose is contained within the calcium alginate gel.

5. The method of claim 1, further comprising co-depositing the source of calcium ions, the source of alginate and a first cell population in the presence of a first electrochemically charged anode under conditions such that a calcium alginate gel containing the first cell population is electrochemically deposited on a first location on the substrate.

6. (canceled)

7. (canceled)

8. The method of claim 5, wherein the first cell population comprises E. coli.

9. The method of claim 8, wherein the E. coli expresses RFP or GFP.

10. The method of claim 5, further comprising co-depositing a source of calcium ions, a source of alginate and a second cell population in the presence of a second electrochemically charged anode under conditions such that a calcium alginate gel containing the second cell population is electrochemically deposited on the substrate in a second location on the substrate.

11. The method of claim 5, further comprising co-depositing agarose, such that the agarose is contained within the calcium alginate gel.

12. The method of claim 11, wherein at least one of the first or second cell populations comprises yeast cells.

13. The method of claim 11, wherein at least one of the first or second cell populations comprises cells comprising a surface expressed protein.

14. The method of claim 13, wherein the surface expressed protein is an antigen receptor.

15. The method of claim 14, wherein the antigen receptor is selected from VLR and an antibody.

16. A method of forming a calcium alginate hydrogel with one or more cell populations on a substrate, comprising co-depositing a source of calcium ions, a source of alginate, agarose and a first cell population in the presence of a first electrochemically charged anode under conditions such that an alginate-agarose gel containing the first cell population is electrochemically deposited on a first location on the substrate.

17. (canceled)

18. (canceled)

19. (canceled)

20. The method of claim 1, further comprising co-deposition with a stimuli-responsive polymer responsive to at least one of: thermal changes, pH changes, and electrical signals, such that the stimuli-responsive polymer is contained within the hydrogel.

21. A method of forming a hydrogel on a substrate, comprising co-depositing at least two stimuli-responsive polymers, wherein at least one of the stimuli-responsive polymers is an electroaddressable polymer and wherein a second stimuli-responsive polymer is responsive to at least one of: thermal changes, pH changes, and electrical signals, under conditions such that a hydrogel comprising the at least two stimuli-responsive polymers is electrochemically deposited on the substrate.

22. The method of claim 21, wherein the electroaddressable polymer is selected from alginate, calcium alginate, and chitosan.

23. The method of claim 21, wherein the second stimuli-responsive polymer is selected from agarose and gelatin.

24. (canceled)

25. (canceled)

26. (canceled)

27. (canceled)

28. (canceled)

29. (canceled)

30. (canceled)

31. (canceled)

32. A method of in-film bioprocessing, comprising:
electrodepositing a hydrogel comprising at least two stimuli-responsive polymers, wherein at least one of the stimuli-responsive polymers is an electroaddressable polymer and wherein a second stimuli-responsive polymer is responsive to at least one of: thermal changes, pH changes, and electrical signals, wherein the hydrogel comprises at least a first cell population, wherein the first cell population comprises cells capable of expressing a surface expressed protein;
proliferating the cells in the hydrogel;
inducing expression of the surface expressed protein;
adding an immunoreagent; and
determining occurrence of binding of the immunoreagent and the surface expressed protein, to form an immunocomplex.

33. The method of claim 32, wherein the immunoreagent is an antigen or antibody.

34. The method of claim 33, wherein the immunoreagent is fluorescently labeled.

35. The method of claim 33, wherein the immunoreagent is HEL antigen.

36. The method of claim 32, wherein the immunoreagent is anti-HA.

37. The method of claim 32, wherein the step of adding an immunoreagent further comprises electrophoresis.
